(19) United States
(12) Patent Application Publication
(10) Pub. No.: US 2005/0124883 A1
(43) Pub. Date: Jun. 9, 2005

(54) Adaptive parallel artifact mitigation

(76) Inventor: Thomas J. Hunt, Pelham, NH (US)

Correspondence Address: PHILIPS INTELLECTUAL PROPERTY & STANDARDS, P.O. BOX 3001, BRIARCLIFF MANOR, NY, 10510, US

(21) Appl. No.: 10/990,632

(22) Filed: Nov. 17, 2004

Related U.S. Application Data
(60) Provisional application No. 60/524,019, filed on Nov. 20, 2003.

(51) Int. Cl. A61B 8/00

(52) U.S. Cl. 600/437

(57) ABSTRACT

A method is disclosed herein for adaptively compensating for multi-line receive artifacts in cardiac ultrasound imaging. In accordance with the method, a plurality of transmit beams are transmitted (201) into a subject with a transmitter having a transmit aperture, wherein each of said transmit beams has a plurality of receive beams associated therewith. The plurality of receive beams corresponding to each of the plurality of transmit beams are then received (203), and the extent of any occlusion of the transmit aperture is determined (205). The occlusion is then compensated for (207).

### CROSS REFERENCE TO RELATED CASES

Applicant claims the benefit of Provisional Application Ser. No. 60/524,019, filed 20 Nov. 2003.

The present invention relates generally to ultrasound imaging, and more particularly to methods for eliminating amplitude modulation artifacts in cardiac ultrasound imaging.

Conventional sonography is conducted with the use of diagnostic ultrasound equipment that transmits sound energy into the human body and receives the signals that reflect off of bodily tissues and organs such as the heart, liver, and kidneys. Blood flow patterns may be obtained from Doppler shifts or from shifts in time domain cross correlation functions due to blood cell motion. These shifts produce reflected sound waves that may be generally displayed in a two-dimensional format known as color flow imaging or color velocity imaging. A typical ultrasound system emits pulses over a plurality of paths and converts echoes received from objects on the plurality of paths into electrical signals used to generate ultrasound data from which an ultrasound image can be displayed. The process of obtaining the raw data from which the ultrasound data is produced is typically termed “scanning,” “sweeping,” or “steering a beam”.

Sonography may be performed in real time, which refers to the presentation of ultrasound images in a rapid sequential format as the scanning is being performed. Typically, the scanning that gives rise to the image is performed electronically, and utilizes a group of transducer elements (called an “array”) which are arranged in a line and which are excited by a set of electrical pulses, one pulse per element. The pulses are typically timed to construct a sweeping action.

Signal processing in an ultrasound scanner usually begins with the shaping and delaying of the excitation pulses applied to each element of the array so as to generate a focused, steered and apodized pulsed wave that propagates into the tissue. The characteristics of the transmitted acoustic pulse may be adjusted or “shaped” to correspond to the setting of a particular imaging mode. For example, pulse shaping may include adjusting the length of the pulse for different lines depending on whether the returned echoes are ultimately to be used in B-scan, pulsed Doppler or color Doppler imaging modes. Pulse shaping may also include adjustments to the central frequency which, in modern broadband transducers, can be set over a wide range and may be selected according to the part of the body that is being scanned. A number of scanners also shape the envelope of the pulse (i.e., by making it Gaussian in shape) to improve the propagation characteristics of the resulting sound wave.

Echoes resulting from scattering of the sound by tissue structures are received by all of the elements within the transducer array and are subsequently processed. The processing of these echo signals typically begins at the individual channel or element level with the application of apodization functions, dynamic focusing, steering delays, and other such procedures.

One of the most important elements in signal processing is beam forming. In a transducer array, the beam is focused and steered by exciting each of the elements at a different time so that the resulting sound wave coming from each element will arrive at the intended focal point simultaneously.

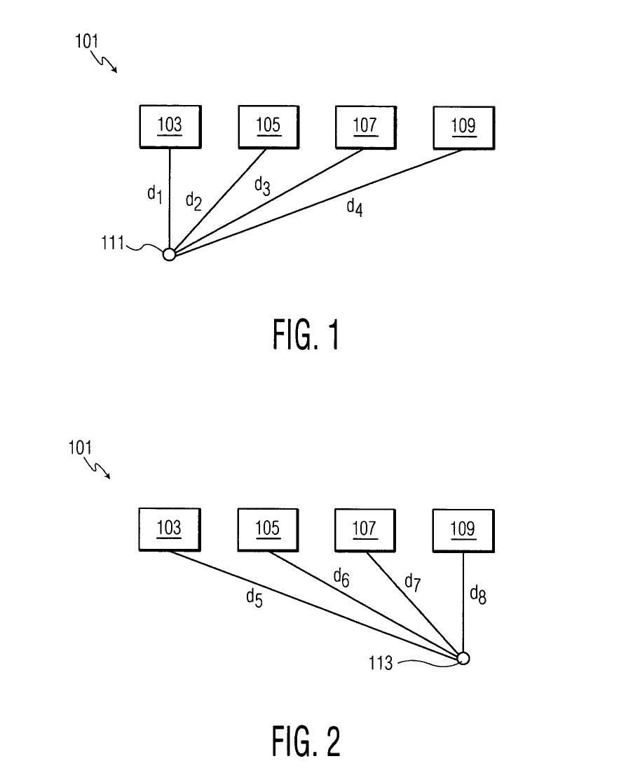

This principle may be understood in reference to FIG. 1, which depicts a transducer array 101 having transducers 103, 105, 107 and 109 that are at distances d₁, d₂, d₃ and d₄, respectively, from focal point 111. In the case depicted, the beam is being focused and steered to the left. Since the distance d₄ from the focal point to transducer element 103 of the transducer array is shorter than the distance d₄ from the focal point to transducer element 109, during transmission, element 109 must be excited before elements 103, 105, and 107 in order for the waves generated by each element to arrive at the focal point simultaneously. By contrast, in the case shown in FIG. 2, the focal point 113 is to the right. Here, the elements of the transducer must be excited in the reverse order during transmission (that is, element 103 must be excited before elements 105, 107, and 109) in order for the waves generated by each element to arrive at the focal point simultaneously. This process of coordinating the firing of transducer elements is referred to as “beam formation”, and the device which implements this process is called a “beam former”.

Beam forming is typically implemented during both transmission (described above) and reception. Beam forming on reception is conceptually similar to beam forming on transmission. On reception, an echo returning from a given point 111 (see FIG. 1) encounters each of the elements 103, 105, 107 and 109 in the transducer array 101 at a different time due to the varying distances d₁, d₂, d₃ and d₄, respectively, of these elements from focal point 111. Consequently, the signals coming into the ultrasound scanner from the various elements must be delayed so that they all “arrive” at the same moment. The signals from each element are then summed together to form the ultrasound signal that is subsequently processed by the rest of the ultrasound instrument. Typically, 1-dimensional arrays having 32 to 192 transducer elements are used for beam formation. The signal from each individual element is delayed in order to steer the beam in the desired direction.

The beam former, in addition to combining the received signals into an output signal, also focuses the beam. When dynamic focusing is used, for each pulse which is transmitted from the array, the beam former tracks the depth and focuses the receive beam as the depth increases. The receive aperture will usually be allowed to increase with depth, since this achieves a lateral resolution which is constant with depth and decreases sensitivity to aberrations in the imaged medium. In order for the receive aperture to increase with depth, it is necessary to dynamically control the number of elements in the array that are used to receive the echoes. Since often a weighting function (apodization) is used to reduce or eliminate side lobes from the combined signal, the element weights also have to be dynamically updated with depth.

Most ultrasound scanners are able to perform parallel beam forming. Parallel beam forming refers to the acquisition of multiple roundtrip beams from a single transmit event by focusing multiple receive beams within a single transmit beam. The transmit beam, due to its single focus, is typically apodized to improve depth of field and is therefore inherently wider than the dynamically focused receive beams. The receive beams have local acoustical maxima which are off-axis relative to the transmit beam. Parallel beam forming allows the imaged field to be scanned faster and thus allows the frames to be updated faster. Parallel beam forming is especially advantageous in 3-D imaging, due to the large number or frames that need to be gathered.

While parallel beam forming has many notable advantages, its application can be significantly complicated by anatomical features. For example, during the imaging of myocardial tissues, the aperture of the phased array transducer is often partially blocked by a rib. Consequently, the resulting transmit beam shifts in location, while the receive beam continues to track the original transmit beam location. This effect causes the roundtrip beam pattern to lose amplitude at all depths other than the transmit focal depth.

During parallel beam forming, the aperture blocking affects each of the parallel roundtrip beams differently, thereby creating “line-to-line” amplitude modulation artifacts. These artifacts cause variations in image brightness which appear as annoying striations running across the image when those beams are placed side by side. In conventional imaging schemes, this problem is frequently dealt with by lateral blending filters that reduce the amplitude differences between lines to produce more uniform brightness. However, this approach causes loss in image resolution.

There is thus a need in the art for a method for effectively reducing or eliminating “line-to-line” amplitude modulation artifacts and other receive artifacts without sacrificing image resolution. There is also a need in the art for such methods that can be employed statically or dynamically. These and other needs are met by the methodologies and devices disclosed herein.

In one aspect, a method for compensating for transmit aperture occlusion in ultrasound imaging is provided. In accordance with the method, a plurality of transmit beams are transmitted into a subject with a transmitter having a transmit aperture, wherein each of said transmit beams has a plurality of receive beams associated therewith. The plurality of receive beams corresponding to each of the plurality of transmit beams are then received, and the extent of any occlusion of the transmit aperture is determined. Various steps may then be taken to compensate for the occlusion, and this compensation may occur statically or dynamically.

The step of compensating for the occlusion may include the step of deactivating both the receive and transmit channels associated with the blocked portion of the aperture. It may also include the step of aligning the center channel focusing coefficient with the new center of the transmit aperture. The extent of occlusion of the transmit aperture may be determined by the amount and location of the amplitude modulation relative to the placement of the receive beams, by monitoring the integrated energy of each receive beam from a point beyond the transmit focus, and/or by firing calibration beams.

In another aspect, a method is provided for compensating for aperture blocking effects in ultrasound imaging of a type that move the original center of the transmit aperture to a new center. In accordance with the method, a plurality of transmit beams is transmitted into a subject, and a plurality of receive beams are received that are associated with each of said transmit beams and that are reflected off of the subject. Each of the receive beams has a receive channel associated therewith. The integrated energy on each of the receive beams is monitored, preferably in real time, from a point beyond the transmit focus, thereby determining the extent of any occlusion. The receive channels associated with any transmit beams blocked by an occlusion are deactivated, and the receive focusing is re-aligned such that the center receive channels are aligned with the new center of the transmit aperture. In some variations, the amount and location of the amplitude modulation relative to the placement of the receive beams is employed as an indicator of the amount and location of the aperture blockage.

In yet another aspect, an ultrasound imaging device is provided which comprises a transducer array having a transmit aperture associated therewith and which emits acoustic pulses over a plurality of transmit channels and which receives echoes of said pulses over a plurality of receive channels. The imaging device further comprises a beam former adapted to determine the extent of any occlusion of the transmit aperture, and to compensate for the occlusion. The beam former may be adapted to (a) monitor the integrated energy on each of the receive beams from a point beyond the transmit focus, thereby determining the extent of any occlusion that moves the original center of the transmit aperture to a new center, (b) deactivate the receive channels associated with any transmit beams blocked by an occlusion, and (c) re-align the receive focusing such that the center receive channels are aligned with the new center of the transmit aperture.

These and other aspects of the teachings herein are described in further detail below.

For a more complete understanding of the present invention and advantages thereof, reference is now made to the following description which is to be taken in conjunction with the accompanying drawings in which like reference numbers indicate like features and wherein:

FIG. 1 is a diagram illustrating the need for time delay to account for differences in the distances between the elements of a transducer array and a focal point in an ultrasound diagnostic system;

FIG. 2 is a diagram illustrating the need for time delay to account for differences in the distances between the elements of a transducer array and a focal point in an ultrasound diagnostic system;

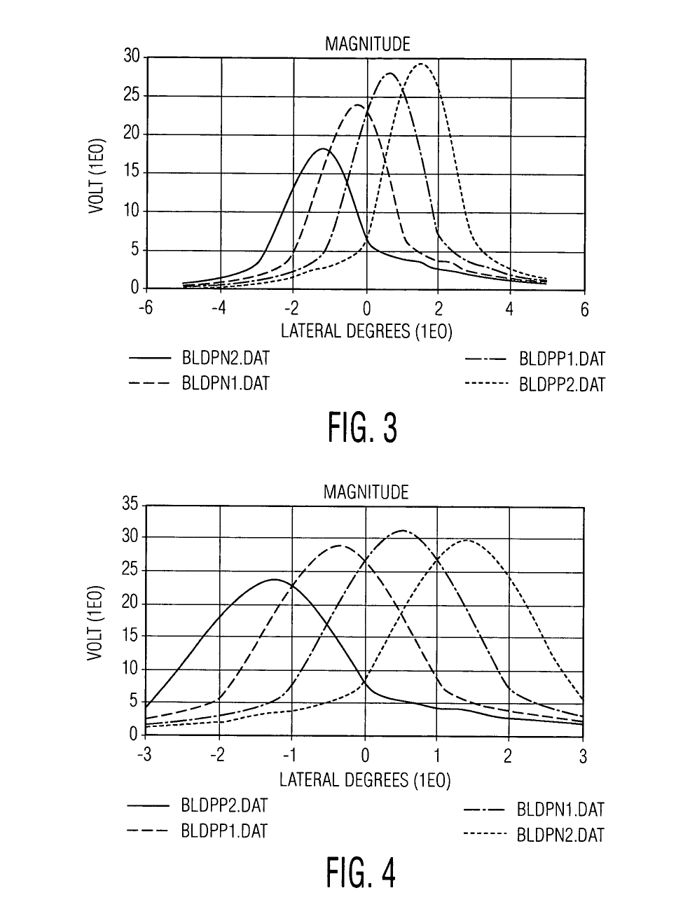

FIG. 3 is a roundtrip beam profile for a 4-way parallel situation and in which a rib blocks the right ⅓ of the total physical aperture;

FIG. 4 is a roundtrip beam profile for a 4-way parallel situation and in which a rib blocks the right ¼ of the total physical aperture;

FIG. 5 is a roundtrip beam profile for a 4-way parallel situation with no rib blockage;

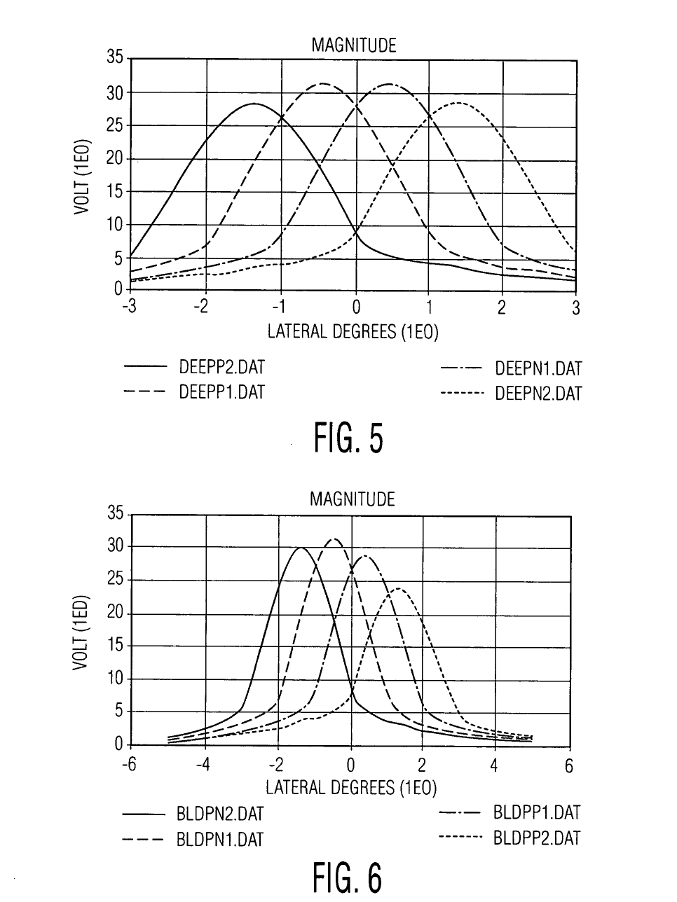

FIG. 6 is a roundtrip beam profile for a 4-way parallel situation and in which a rib blocks the left ¼ of the total physical aperture;

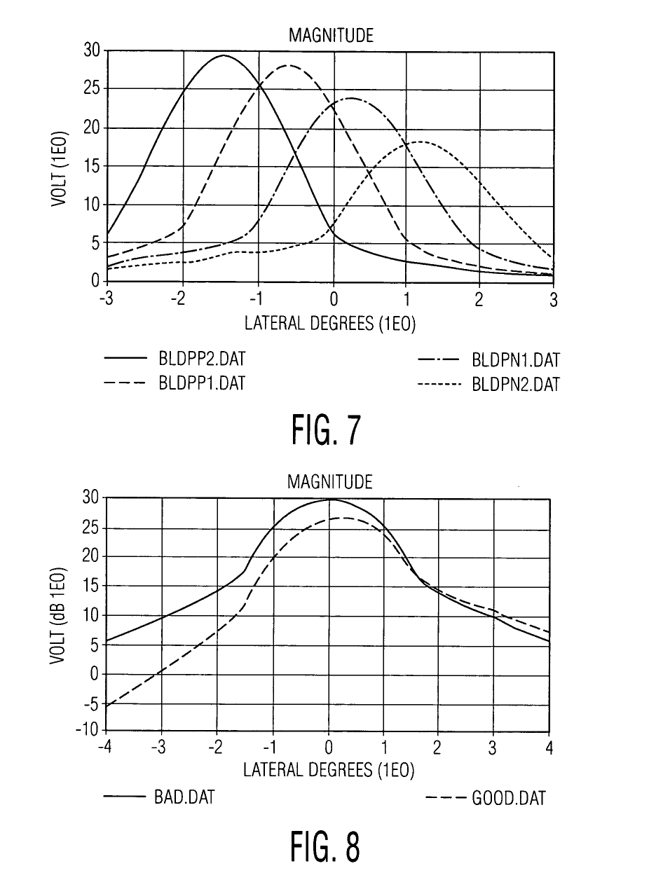

FIG. 7 is a roundtrip beam profile for a 4-way parallel situation and in which a rib blocks the left ⅓ of the total physical aperture;

FIG. 8 is a roundtrip beam profile for a non-parallel case illustrating the drop in amplitude of the roundtrip beam away from the transmit focus and the creation of an asymmetric side lobe pattern;

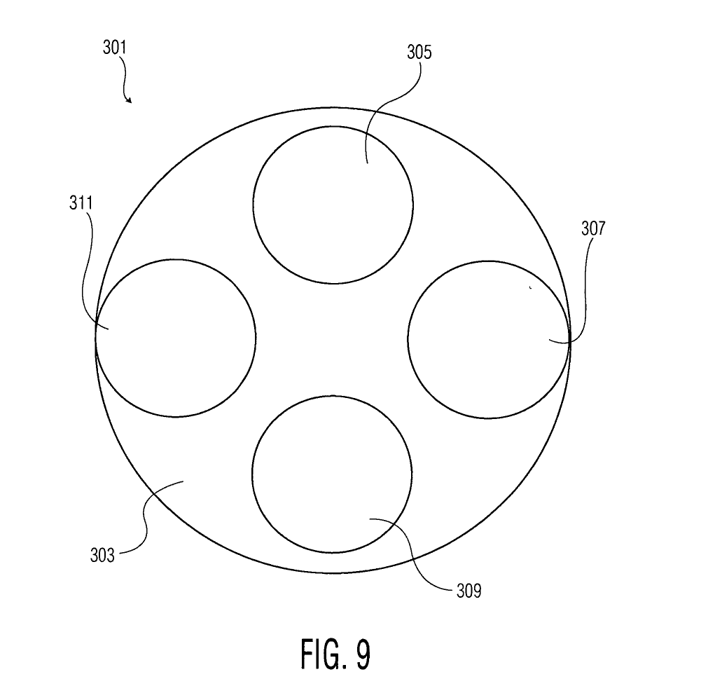

FIG. 9 is an illustration of a 4-way parallel beam pattern on the receive side;

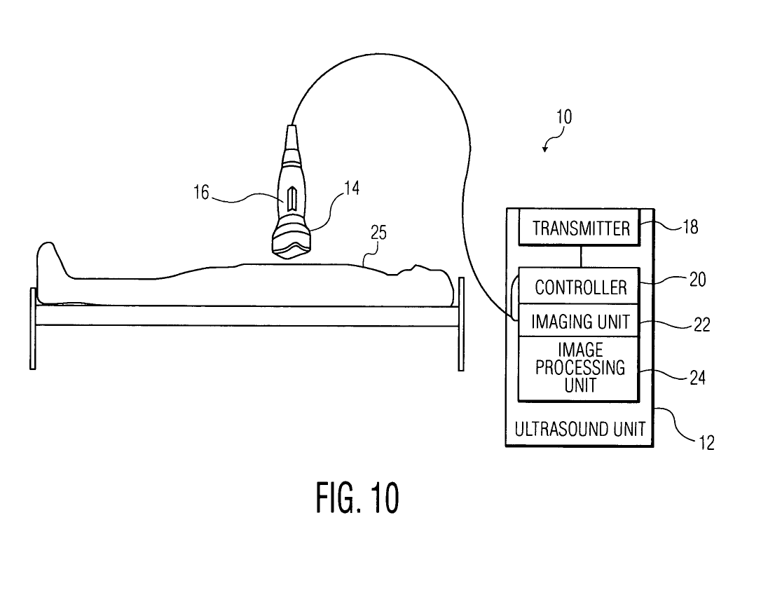

FIG. 10 is an illustration of an ultrasound device which may be used to implement the methodologies disclosed herein;

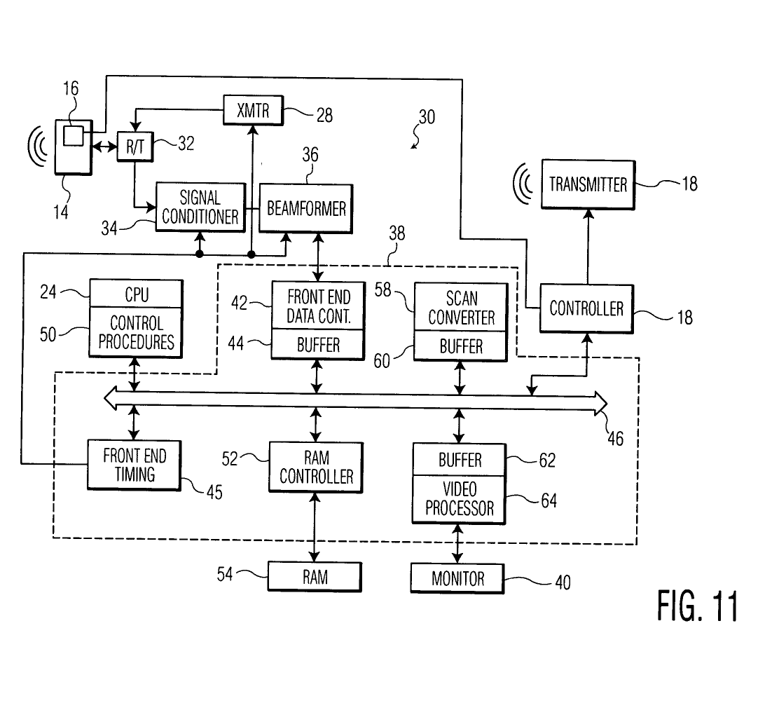

FIG. 11 is a schematic diagram illustrating the functional elements of a device of the type depicted in FIG. 10;

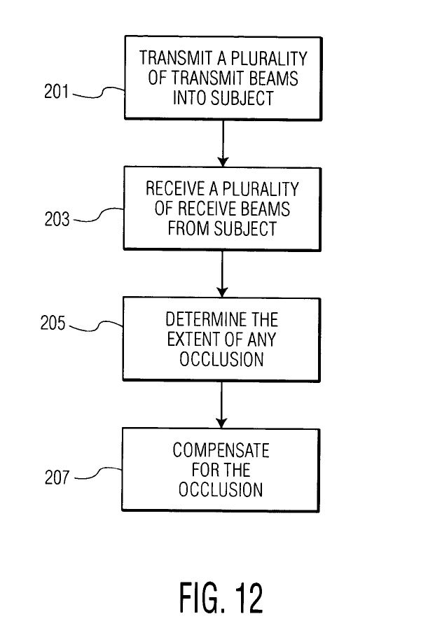

FIG. 12 is a flow chart illustrating one embodiment of the methodology disclosed herein; and

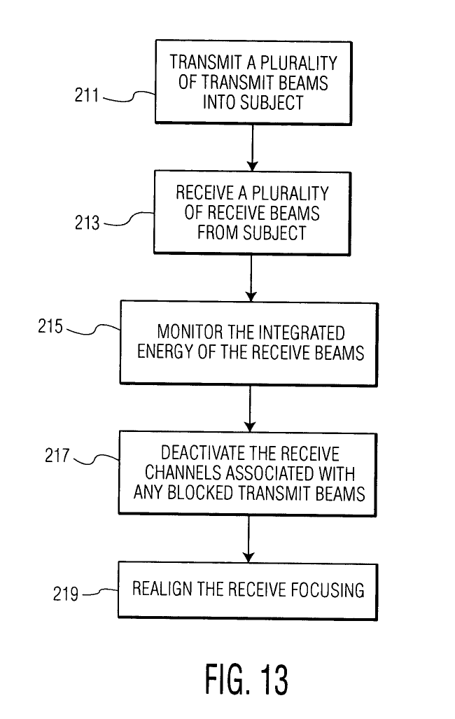

FIG. 13 is a flow chart illustrating another embodiment of the methodology disclosed herein.

The preferred embodiment of the present invention and its advantages are best understood by referring to FIGS. 1 through 9, like numerals being used for like and corresponding parts of the various drawings.

In accordance with the teachings herein, methods for adaptively compensating for “line-to-line” amplitude modulation artifacts in ultrasound imaging, and devices for employing these methods, are provided herein. The methods, which may be static or dynamic and which may be employed in parallel or non-parallel systems, utilize a detection scheme for detecting the presence of an occlusion, and a correction scheme for compensating for the presence of the occlusion.

One embodiment of the detection/correction algorithms disclosed herein may be understood generally with reference to FIG. 12. In the approach depicted therein, a plurality of transmit beams are transmitted 201 into a subject with a transmitter having a transmit aperture, wherein each of said transmit beams has a plurality of receive beams associated therewith. The plurality of receive beams corresponding to each of the plurality of transmit beams are then received 203, the extent of any occlusion of the transmit aperture is determined 205, the occlusion is compensated for 207.

Another embodiment of the detection/correction algorithms disclosed herein may be understood generally with reference to FIG. 13. In the approach depicted therein, a method is provided for compensating, in ultrasound imaging, for aperture blocking effects of a type that move the original center of the transmit aperture to a new center. In accordance with the method, a plurality of transmit beams are transmitted 211 into a subject, and a plurality of receive beams which are associated with each of the transmit beams and which are reflected off of the subject are received 213. Each of the receive beams has a receive channel associated therewith. The integrated energy on each of the receive beams is monitored 215 from a point beyond the transmit focus, thus allowing the extent of any occlusion to be determined. The receive channels associated with any transmit beams blocked by an occlusion are then deactivated 217, and the receive focusing is realigned 219 such that the center receive channels are aligned with the new center of the transmit aperture.

The methodologies disclosed herein may be further understood with reference to FIGS. 3-7, which illustrate 5 cases of aperture blockage and their resulting roundtrip beam profiles for a 4-way parallel situation. In each case, the beam plots were taken at a depth of 8 cm with a transmit focus of 4 cm, and a heavily apodized transmit aperture was run to broaden the transmit beam to allow 4-way parallel beam forming. TABLE 1 below illustrates the affect on roundtrip beam intensity for each of 4 angles of interrogation for five different aperture blockage situations. Case 3, depicted in FIG. 5, represents the expected “symmetric about center” pattern associated with no aperture blockage. The effects summarized in TABLE 1 and illustrated in FIGS. 3-7 would be more pronounced with an integrated measurement over multiple depths beyond the transmit focus.

TABLE 1
Aperture Blocking Effects

| | | | Portion | | | | |
|---|---|---|---|---|---|---|---|
| | | | of | | | | |
| | | Portion of | Active | Rec. 1 | Rec. 2 | Rec. 3 | Rec. 4 |
| | | Physical | Aper- | Peak | Peak | Peak | Peak |
| | | Aperture | ture | Amp. | Amp. | Amp. | Amp. |
| Case | FIG. | Blocked | Blocked | (Volt) | (Volt) | (Volt) | (Volt) |
| 1 | 3 | Right ⅓ | ⅙ | 18 | 24 | 28 | 29* |
| 2 | 4 | Right ¼ | ⅛ | 24 | 29 | 31* | 30 |
| 3 | 5 | None | None | 28 | 31* | 31* | 28 |
| 4 | 6 | Left ¼ | ⅛ | 30 | 32* | 29 | 24 |
| 5 | 7 | Left ⅓ | ⅙ | 29* | 28 | 24 | 19 |

*represents location of transmit beam maximum

As seen from the results set forth in TABLE 1 and illustrated in FIGS. 3-7, the occlusion of the active aperture by the ribs results in amplitude modulation of the receive beams. In accordance with some of the methods disclosed herein, the amount and location of this amplitude modulation relative to the placement of the receive beams is used as an indicator of the amount and location of the aperture blockage. Placement of time gated accumulators within each of the parallel signal paths would permit monitoring of the integrated energy on each of the receive beams from a point beyond the transmit focus (it can be shown that there is no change in the relative beam magnitudes at the focal depth since the transmit beam is in the proper location at the focal depth, and only at that depth). Hence, a detection scheme can be implemented that uses this information to determine the degree and location of occlusion. Once the nature of the occlusion is determined, various correction schemes, including both static and dynamic correction schemes, can be employed to compensate for the presence of the occlusion.

1. Static Correction Schemes

In one static correction scheme that may be employed in accordance with the teachings herein, after the extent of occlusion has been determined, the system turns off both the receive and transmit channels determined to be occluded. The receive focusing parameters are then adjusted so that the center channel focusing coefficients are aligned with the new center of the transmit aperture. Hence, if it is determined that a portion of the active aperture is occluded, the active aperture can be translated over so that it is re-centered about the non-occluded portion of the original active aperture, after which scanning can resume. This process effectively redefines the active aperture by making appropriate steering angle adjustments, and the transmit beam is focused from the new active aperture. TABLE 1 illustrates how such a process might be implemented for the five detection cases mentioned above and illustrated in FIGS. 3-7.

TABLE 2
Static Correction Scheme

| | | | | Extent of re- |
|---|---|---|---|---|
| | | Channels | Direction | alignment |
| | | Turned | of re- | (# channel |
| Case | FIG. | Off | alignment | positions) |
| 1 | 3 | 85-127 | Left | 10 |
| 2 | 4 | 96-127 | Left | 5 |
| 3 | 5 | None | None | None |
| 4 | 6 | 0-31 | Right | 5 |
| 5 | 7 | 0-41 | Right | 10 |

By re-alignment of the active aperture, “line-to-line” amplitude modulation artifacts are eliminated, because the modified receive beams now track the transmit beam correctly. Consequently, image brightness is much more uniform. Moreover, any drop in resolution comes from the aperture occlusion itself, not from the re-alignment of the focus. Hence, this correction scheme does not itself result in any further loss of image resolution. By contrast, the conventional approach of using lateral blending filters to reduce amplitude differences between lines (thereby producing an image of more uniform brightness and eliminating image striations) results in loss of image resolution in addition to the loss caused by the occlusion itself.

Several variations on this approach are possible in accordance with the teachings herein. For example, re-alignment of the receive focusing could be done by re-mapping the receive focusing coefficients to receiver channel assignments. Alternatively, the receive focusing coefficients could be pre-calculated for various states of occlusion, stored off-line, and then accessed as needed.

2. Dynamic Correction Scheme

In the methods described above, the correction scheme is static. That is, the probe is placed over the area to be imaged, and adjustments are made if an occlusion is present. These adjustments may occur automatically, or through a suitable prompt (e.g., by pressing a button on the probe). In many instances, however, a dynamic scheme is required. For example, since the sonographer typically moves the transducer array somewhat continuously during an exam, it is desirable to be able to handle movement from a fully open acoustic window (i.e., no occlusion) to a partially blocked acoustic window, and then either back to a fully open acoustic window or to a more severely blocked acoustic window.

Various dynamic adaptive algorithms can be employed in accordance with the teachings herein to account for the presence of occlusions in such situations. Some of these dynamic algorithms are adaptations of the static schemes described herein.

For example, in the 4-way case illustrated in FIGS. 3-7, the correction algorithm can simply constrain itself to moving from one case (of a static correction scheme) to the next in a continuous, closed loop format. Some hysteresis may be employed to ensure smooth imaging. Consequently, the steady state in such a scheme would be the case illustrated in FIG. 3, in which the amplitudes are symmetric about the optical axis.

3. Extension for Non-Parallel Systems

The same misalignment of the transmit and receive beams also occurs in the non-parallel case (that is, in cases where the transducers in the transducer array are not arranged in parallel), although it does not typically manifest itself with the “line-to-line” artifact that occurs in the image when a parallel beam former is used. Instead, the misalignment is manifested as a drop in amplitude of the roundtrip beam away from the transmit focus and by the creation of an asymmetric side lobe pattern. This effect is illustrated in FIG. 8.

In the non-parallel case, the system can be adapted to fire calibration lines to determine the location of the transmit beam, each with a different receive angle. The resulting roundtrip integrated energies may then be compared in order to determine the extent of the occlusion. These calibration lines can be shot, for example, prior to each acoustic frame (e.g., at 1/30 second intervals). A similar correction scheme can be employed to correct for misaligned beams.

4. Extension to 3-D Imaging with a Matrix Transducer

The methods disclosed herein have principally been described with reference to 2-D imaging. However, these methods may be readily adapted to 3-D imaging. To do so, the detection scheme would need to track the rib placement in 2 dimensions, and the correction scheme would need to shift the center of the receiver focus in 2.dimensions as well.

FIG. 9 illustrates a 4-way parallel beam pattern 301 that can be employed in 3-D imaging. The beam pattern includes a transmit beam 303 and receive beams 305, 307, 309 and 311. In order to determine which way to adjust the center of each receive aperture in the elevation direction, the integrated energy of receive beams 305 and 309 is compared. Similarly, in order to determine which way to adjust the center of each receive aperture laterally, the integrated energy of receive beams 307 and 311 is compared. In most other respects, a static or dynamic correction scheme employed in a 3-D setting would be conceptually similar to a scheme employed in a 2-D setting.

FIG. 10 shows a simplified block diagram of one possible ultrasound imaging system 10 that may be used in the implementation of the methodologies disclosed herein. It will be appreciated by those of ordinary skill in the relevant arts that the ultrasound imaging system 10, as illustrated in FIG. 10, and the operation thereof as described hereinafter, is intended to be generally representative of such systems and that any particular system may differ significantly from that shown in FIG. 10, particularly in the details of construction and in the operation of such system. As such, the ultrasound imaging system 10 is to be regarded as illustrative and exemplary, and not limiting, as regards the methodologies and devices described herein or the claims attached hereto.

The ultrasound imaging system 10 generally includes an ultrasound unit 12 and a connected transducer 14. The transducer 14 includes a spatial locator receiver 16. The ultrasound unit 12 has integrated therein a spatial locator transmitter 18 and an associated controller 20. The controller 20 provides overall control of the system by providing timing and control functions. The control routines include a variety of routines that modify the operation of the receiver 16 so as to produce a volumetric ultrasound image as a live real-time image, a previously recorded image, or a paused or frozen image for viewing and analysis.

The ultrasound unit 12 is also provided with an imaging unit 22 for controlling the transmission and receipt of ultrasound, and an image processing unit 24 for producing a display on a monitor (See FIG. 11). The image processing unit 24 contains routines for rendering a three-dimensional image. The transmitter 18 is preferably located in an upper portion of ultrasound unit 12 so as to obtain a clear transmission to the receiver 16. Although not specifically illustrated, the ultrasound unit described herein may be configured in a cart format.

During freehand imaging, a technician moves the transducer 14 over the subject 25 in a controlled motion. The ultrasound unit 12 combines image data produced by the imaging unit 22 with location data produced by the controller 20 to produce a matrix of data suitable for rendering onto a monitor (see FIG. 11). The ultrasound imaging system 10 integrates image rendering processes with image processing functions using general purpose processors and PC-like architectures. On the other hand, use of ASICs to perform the stitching and rendering is possible.

FIG. 11 is a block diagram 30 of an ultrasound system that may be used in the practice of the methodologies disclosed herein. The ultrasound imaging system shown in FIG. 11 is configured for the use of pulse generator circuits, but could be equally configured for arbitrary waveform operation. The ultrasound imaging system 10 uses a centralized architecture suitable for the incorporation of standard personal computer (“PC”) type components and includes a transducer 14 which, in a known manner, scans an ultrasound beam, based on a signal from a transmitter 28, through an angle. Backscattered signals or echoes are sensed by the transducer 14 and fed, through a receive/transmit switch 32, to a signal conditioner 34 and, in turn, to a beam former 36. The transducer 14 includes elements which are preferably configured as a steerable, two-dimensional array. The signal conditioner 34 receives backscattered ultrasound signals and conditions those signals by amplification and forming circuitry prior to their being fed to the beam former 36. Within the beam former 36, ultrasound signals are converted to digital values and are configured into “lines” of digital data values in accordance with amplitudes of the backscattered signals from points along an azimuth of the ultrasound beam.

The beam former 36 feeds digital values to an application specific integrated circuit (ASIC) 38 which incorporates the principal processing modules required to convert digital values into a form more conducive to video display that feeds to a monitor 40. A front end data controller 42 receives lines of digital data values from the beam former 36 and buffers each line, as received, in an area of the buffer 44. After accumulating a line of digital data values, the front end data controller 42 dispatches an interrupt signal, via a bus 46, to a shared central processing unit (CPU) 48. The CPU 48 executes control procedures 50 including procedures that are operative to enable individual, asynchronous operation of each of the processing modules within the ASIC 38. More particularly, upon receiving an interrupt signal, the CPU 48 feeds a line of digital data values residing in a buffer 42 to a random access memory (RAM) controller 52 for storage in random access memory (RAM) 54 which constitutes a unified, shared memory. RAM 54 also stores instructions and data for the CPU 48 including lines of digital data values and data being transferred between individual modules in the ASIC 38, all under control of the RAM controller 52.

The transducer 14, as mentioned above, incorporates a receiver 16 that operates in connection with a transmitter 28 to generate location information. The location information is supplied to (or created by) the controller 20 which outputs location data in a known manner. Location data is stored (under the control of the CPU 48) in RAM 54 in conjunction with the storage of the digital data value.

Control procedures 50 control a front end timing controller 45 to output timing signals to the transmitter 28, the signal conditioner 34, the beam former 36, and the controller 20 so as to synchronize their operations with the operations of modules within the ASIC 38. The front end timing controller 45 further issues timing signals which control the operation of the bus 46 and various other functions within the ASIC 38.

As previously noted, control procedures 50 configure the CPU 48 to enable the front end data controller 44 to move the lines of digital data values and location information into the RAM controller 52, where they are then stored in RAM 54. Since the CPU 48 controls the transfer of lines of digital data values, it senses when an entire image frame has been stored in RAM 54. At this point, the CPU 48 is configured by control procedures 50 and recognizes that data is available for operation by a scan converter 58. At this point, therefore, the CPU 48 notifies the scan converter 58 that it can access the frame of data from RAM 54 for processing.

To access the data in RAM 54 (via the RAM controller 52), the scan converter 58 interrupts the CPU 48 to request a line of the data frame from RAM 54. Such data is then transferred to a buffer 60 associated with the scan converter 58 and is transformed into data that is based on an X-Y coordinate system. When this data is coupled with the location data from the controller 20, a matrix of data in an X-Y-Z coordinate system results. A four-dimensional matrix may be used for 4-D (X-Y-Z-time) data. This process is repeated for subsequent digital data values of the image frame from RAM 54. The resulting processed data is returned, via the RAM controller 52, into RAM 54 as display data. The display data is typically stored separately from the data produced by the beam former 36. The CPU 48 and control procedures 50, via the interrupt procedure described above, sense the completion of the operation of the scan converter 58. The video processor 62 interrupts the CPU 48 which responds by feeding lines of video data from RAM 54 into the buffer 62, which is associated with the video processor 64. The video processor 64 uses video data to render a three-dimensional volumetric ultrasound image as a two-dimensional image on the monitor 40.

The above description of the invention is illustrative, and is not intended to be limiting. It will thus be appreciated that various additions, substitutions and modifications may be made to the above described embodiments without departing from the scope of the present invention. Accordingly, the scope of the present invention should be construed solely in reference to the appended claims.

1. A method for compensating for transmit aperture occlusion in ultrasound imaging, comprising the steps of:
transmitting a plurality of transmit beams into a subject with a transmitter having a transmit aperture, wherein each of said transmit beams has a plurality of receive beams associated therewith;
receiving the plurality of receive beams corresponding to each of the plurality of transmit beams;
determining the extent of any occlusion of the transmit aperture; and
compensating for the occlusion.

2. The method of claim 1, wherein each transmit beam has at least one transmit channel and at least one receive channel associated therewith, and wherein the step of compensating for the occlusion includes the step of deactivating the receive and transmit channels blocked by the occlusion.

3. The method of claim 1, wherein each transmit beam has at least one transmit channel and at least one receive channel associated therewith, wherein the occlusion causes the transmit aperture to have a new center distinct from its original center, and further comprising the steps of:
determining the new center of the transmit aperture; and
aligning the center receive channels with the new center of the transmit aperture.

4. The method of claim 1, wherein the extent of occlusion of the transmit aperture is determined from the amount and location of any roundtrip amplitude modulation relative to the placement of the receive beams.

5. The method of claim 1, wherein the extent of occlusion is determined by monitoring the integrated energy of the receive beams from a point beyond the transmit focus.

6. The method of claim 1; wherein the step of compensating for any occlusion of the transmit aperture includes the step of refocusing the transmit beam.

7. The method of claim 12 wherein the extent of any occlusion is determined by monitoring the peak amplitudes of the receive beams.

8. The method of claim 1, wherein the step of determining the extent of any occlusion of the transmit aperture includes the step of firing calibration beams.

9. The method of claim 1, wherein the step of compensating for any occlusion is static.

10. The method of claim 1, wherein the step of compensating for any occlusion is dynamic.

11. A method for compensating for aperture blocking effects in ultrasound imaging of a type that move the original center of the transmit aperture to a new center, the method comprising the steps of:
transmitting a plurality of transmit beams into a subject;
receiving a plurality of receive beams associated with each of said transmit beams and reflected off of the subject, each of said receive beams having a receive channel associated therewith;
monitoring the integrated energy on each of the receive beams from a point beyond the transmit focus, thereby determining the extent of any occlusion;
deactivating the receive channels associated with any transmit beams blocked by an occlusion; and
re-aligning the receive focusing such that the center receive channels are aligned with the new center of the transmit aperture.

12. The method of claim 11, wherein the monitoring occurs in real time.

13. The method of claim 11, wherein the amount and location of the amplitude modulation relative to the placement of the receive beams is employed as an indicator of the amount and location of the aperture blockage.

14. An ultrasound imaging device, comprising:
a transducer array which emits acoustic pulses over a plurality of transmit channels and which receives echoes of said pulses over a plurality of receive channels, said transducer array having a transmit aperture associated therewith; and
a beam former adapted to (a) determine the extent of any occlusion of the transmit aperture, and (b) compensate for the occlusion.

15. The ultrasound imaging device of claim 14, wherein said beam former is adapted to (a) monitor the integrated energy on each of the receive beams from a point beyond the transmit focus, thereby determining the extent of any occlusion that moves the original center of the transmit aperture to a new center, (b) deactivate the receive channels associated with any transmit beams blocked by an occlusion, and (c) re-align the receive focusing such that the center receive channels are aligned with the new center of the transmit aperture.
